(19) United States
(12) Patent Application Publication
(10) Pub. No.: US 2006/0228337 A1
(43) Pub. Date: Oct. 12, 2006

(54) Use of bacilli bacteria in order to produce a composition for the prevention of vertebral compression syndrome in salmonids

(75) Inventors: Joël Aubin, (US); Laurent Labbe, Sizun (FR); Francois-Joäl Gatesoupe, Trebabu (FR); Luc Lebrun, Pont De Buis (FR)

Correspondence Address: YOUNG & THOMPSON, 745 SOUTH 23RD STREET, 2ND FLOOR, ARLINGTON, VA, 22202, US

(73) Assignees: Institute Francais de Recherche Pour L-explotation de la mer, Issy les Moulineaux (FR); Institute National de la Recherche Agronomique-Inra, -Paris Cedex 07 (FR)

(21) Appl. No.: 10/562,204

(22) PCT Filed: Jun. 23, 2004
(86) PCT No.: PCT/FR04/50288

§ 371 Date: Apr. 14, 2006

(30) Foreign Application Priority Data
Jun. 24, 2003 (FR) 03/50251

(51) Int. Cl. A61K 35/74

(52) U.S. Cl. 424/93.4; 424/93.45

(57) ABSTRACT

The invention relates to the use of cells of at least one strain of bacteria belonging to the Bacilli class for the production of a composition that is intended to prevent vertebral compression syndrome in fish from the salmonid family.

The present invention relates to the field of the prophylaxis of the vertebral compression syndrome in fish belonging to the salmonid family.

There is nowadays an increasing human consumption of fish belonging to the salmonid family, which comprises, among others, salmon, trout and char. In order to be able to offer to the final consumer salmonid fish with a moderate price, such fish are from now on produced on an industrial scale, via aquaculture, in fish breeding farms. So, about 53,000 metric tons of fresh fish originating from fish farming were produced in France in the year 2002. Amongst the salmonids, trout accounts for 77% of fresh fish produced in French fish breeding farms.

In order to constantly improve the economic conditions of the production of fish farming fresh fish, producers are continuously making progress in the sanitary and food conditions in farms, with the aim to offer to the final consumer fresh fish with a high sanitary and gustatory quality. The farm yield has also been considered and producers try to reduce, as much as possible, the death rate and the impact of various pathologies in fish, more particularly in fish being in growth phase.

In fish farming grown salmonids, in particular for rainbow trout, a high proportion of subjects is observed as being suffering from vertebral compression syndrome, which is a skeleton disease resulting in (i) a malformation of some vertebrae and (ii) some vertebrae fusing together, said skeleton malformations being sometimes distributed in the front region, the pre-caudal and the caudal region of the salmonid spinal column.

Although the vertebral compression syndrome does not affect the salmonid sanitary or gustatory quality, such pathology leads to a skeleton deformity such that the fish is unsuitable for being offered to the final consumer. In some salmonids, such as rainbow trout, said disease could affect up to 30% of the fish being grown in the fish breeding facilities. The impact level of the vertebral compression syndrome in fish farming salmonids considerably decreases the industrial production yield and has, accordingly, a direct influence on the cost price of the fresh fish being marketed.

Practically, producers have no other solution than eliminating, through a simple naked eye inspection, individuals suffering from the vertebral compression syndrome. Such a selection is all the more costly for farms as the visual inspection of such skeleton malformations is only possible when the fish are in the growth final phase.

Nowadays, the only known treatment allowing for reducing the vertebral compression syndrome impact is a preventive antibiotic treatment based on the active compound florphenicol. Treating young trout for 10 to 20 days using such an antibiotic should reduce by more than 60% the proportion of salmonids suffering from such a disease.

However, making use of such an antibiotic for a preventing purpose in fish farming facilities has several disadvantages. More particularly, extensively using antibiotics, especially florphenicol, is likely to cause the development and the dissemination of an anti-bioresistance, for example through plasmid transmission, within the microbial flora present in fish breeding farms. It goes without saying that the development of a resistance to florphenicol, being a broad activity spectrum antibiotic, would make it impossible for the producer to use any prophylactic or therapeutic treatment in the case of a subsequent bacterial infection in the farm. Moreover, an antibiotic treatment could only be used after a farm preliminary veterinary diagnosis, followed by a specific prescription from a veterinary surgeon.

The antibiotic remains present, even in a trace state, in the fish flesh being ingested by the final consumer, which is likely to cause an anti-bioresistance phenomenon also in the general human population.

In addition, using an antibiotic, such as florphenicol, could lead to a growth delay as well as an increase in the mortality rate of young fish (alevin) compared to the untreated young fish.

Finally, regulations relating to breeding conditions of fish farming fish are more and more stringent, and could, consequently, in a near future, prohibit resorting at all to the use of an antibiotic.

It would therefore be desirable to find an alternative to the present prophylactic treatments of the vertebral compression syndrome in fish farming salmonids, which, more particularly, would avoid resorting at all to the use of an antibiotic pharmaceutical composition and which, preferably, would not comprise a drug based treatment.

Such objectives have been achieved according to the invention.

It has been shown according to the invention that a composition containing cells of at least one bacterium strain of the Bacilli class mainly used as a food additive, allows to reduce the impact of the vertebral compression syndrome in fish farms, with at least the same efficiency as the florphenicol antibiotic.

In addition, it has been shown according to the invention that the prophylactic supply of a composition containing cells of at least one bacterium strain of the Bacilli family to fish farming salmonids has no impact on the survival rate of young fish, compared to a young fish group which did not receive such a prophylactic supply.

Moreover, the prophylactic supply of bacteria of the Bacilli class does not result in any decrease of the growth of fish farming salmonids.

The object of the present invention is the use of cells of at least one bacterium strain of the Bacilli family for preparing a composition intended for preventing the vertebral compression syndrome in fish belonging to the salmonid family.

The vertebral compression syndrome, that could be visually detected by means of salmonid apparent malformations in the growth final phase, comprises two skeleton alteration types, respectively (i) a decrease in the width of some vertebrae compared to the normal width and (ii) some vertebrae fusing together, in one or more regions of the spinal column, such skeleton alterations being able to be detected and visualized through X ray radiography.

FIG. 1A shows an X ray radiophotography of part of the spinal column of a salmonid with a normal skeleton growth.

FIG. 1B shows an X ray radiophotography of part of the spinal column of a salmonid suffering from a vertebral compression syndrome.

As may be seen in FIG. 1, the vertebral compression syndrome leads to a decrease of the vertebra width, more particularly for vertebrae being fused together. Depending on the severity of the vertebral compression syndrome, i.e., depending on the number of affected vertebrae, such a disease induces a more or less high overall decrease in the fish length and, as a function of the spinal column areas being affected, also to a growth distortion. In addition, such skeleton distortions are coupled with an alteration of the bone structure. More particularly, the fused vertebrae show a compactness (bone surface/overall surface) significantly higher than vertebrae of non affected fish.

The skeleton malformations of salmonids developing a vertebral compression syndrome are essentially frequent in three areas of the spinal column: (i) a front region between the V8 vertebra and the V21 vertebra, (ii) a pre-caudal region between the V31 vertebra and the V40 vertebra and (iii) a caudal region between the V51 vertebra and the V61 vertebra.

Said third skeleton region, being located in the urophore complex, in the vicinity of the caudal fin, mainly has malformed vertebrae but very few fused vertebrae.

As used herein, <<salmonid>> means teleost bone fish of the Oncorhynchus, Salmo, Salvenius, Hucho, Thymallus, Coregonus species, sharing in common numerous anatomical and physiological characteristics, such as, more particularly, an identical digestive system comprising an oesophagus, a stomach, pyloric caeca and a short intestine. Also, all the salmonids have, between the dorsal and caudal fins, a so-called <<adipose fin>> small fin.

The common anatomical and physiological characteristics of the digestive system of all salmonids allow to easily switch from one salmonid species to another an efficient prophylactic treatment based on Bacilli cells coming in contact first with the various parts of the digestive system and being consequently biologically active in said part of the organism.

As used herein, <<salmonid>> means teleost fish of the salmon family, such as salmons, trout, chars, graylings and coregons.

Are included in the salmon type fish of the Salmo salar L, Oncorhynchus tshawytscha (chinook salmon), Oncorhynchus gorbuscha (pink salmon), Oncorhynchus nerka (kokanee salmon), Oncorhynchus keta (keta salmon) and Oncorhynchus kisutch (coho salmon) species.

Are also included in the salmonid family, the various trout, including Salmo trutta L. (sea trout), Salmo fario (river trout), Salmo lacustris (lake trout), Cristivomer namaycush (Canadian trout), or Oncorhynchus mykiss (rainbow trout), Oncorhynchus clarki (made-up trout).

Are also part of the salmonids, fish of the char fish type such as Salvelinus alpinus (artic char) as well as Salvelinus fontinalis (fountain salmon).

Also included in salmonids are fish of the grayling type such as Thymallus thymallus (river grayling).

Also included in salmonids are fish of the coregon type, such as Coregonus fera (fera coregon), Coregonus hiemalis (gravenche), Coregonus wartmanni (lavaret), Coregonus macrophthalmus (bondelle), Coregonus coregonus schinzi (palee).

Also included in salmonids are Huchon fish of Hucho hocho type.

Preferably, cells are used of at least one bacterium strain of the Bacilli class for preparing a composition comprising 10³ to 10⁸ colony forming units (cfu) of said bacterium of the Bacilli class per gram of the composition.

Preferably, the prophylactic composition is used, such as defined hereinabove, in amounts corresponding to a number of colony forming units (cfu) comprised between 10⁹ and 10¹³ cfu per metric ton of salmonids per day.

As used herein, <<Bacilli>> means bacteria as described in Bergey's handbook and belonging to class III of Firmicute's phylum (Taxonomic outline of the procaryotes, Bergey's Manual of Systematic Bacteriology, 2^{nd} edition, July 2002, Georges M. Garrity, Kristin L. Johnson, Julia Bell and Denise B. Searles, Springer-Verlag, New York, p. 153-181).

More specifically, <<Bacilli>> means, as used herein, bacteria of the Bacillus-Lactobacillus-Streptococcus group (Tax ID 91061, NCBI, http://www.ncbi.nlm.nih.gov/Taxonomy/taxonomyhome.html). Bacteria of the Bacillaceae, Lactobacillaceae, Carnobacteriaceae, Enterococcaceae, Leuconostocaceae, and Streptococcacea families are preferred. Preferably, strains could be used such as Bacillus subtilis ATCC 6051, Bacillus cereus ATCC 14579, Bacillus megaterium ATCC 14581, Bacillus polymyxa ATCC 842, Bacillus licheniformis ATCC 14580, Lactobacillus rhamnosus ATCC 7469, Lactobacillus plantarum ATCC 14917, Lactobacillus casei ATCC 393, Lactobacillus acidophilus ATCC 4356, Lactobacillus johnsonii ATCC 33200, Lactobacillus helveticus ATCC 15009, Lactobacillus bulgaricus ATCC 11842, Lactobacillus fructivorans ATCC 8288, Lactobacillus brevis ATCC 14869, Pediococcus acidilactici DSM 20284, Pediococcus pentosaceus ATCC 33316, Carnobacterium divergens ATCC 35677, Carnobacterium inhibens CCUG 31728, Enterococcus faecium ATCC 19434, Leuconostoc cremoris ATCC 19254, Weissella hellenica ATCC 51523, Streptococcus thermophilus ATCC 19258, Lactococcus lactis ATCC 19435.

A prophylactic probiotic composition used according to the invention could comprise cells of one single bacterium strain of the Lactobacillaceae family such as hereinafter defined, or also several strains of such bacteria, in association.

When several bacterium strains of the Lactobacillaceae family are present in association in a prophylactic composition according to the invention, are preferably combined at the most 2, 3, 4 or 5 distinct strains, undiscriminately belonging to the Bacillus, Lactobacillus, Pediococcus, Carnobacterium, Enterococcus, Leuconostoc, Weissella, Streptococcus, Lactococcus species.

Preferably, for preparing a prophylactic composition according to the invention, cells are used of a bacterium strain of the Lactobacillaceae family selected amongst Lactobacillus rhamnosus CNCM MA 543/2B, or Lactobacillus plantarum CNCM MA 18/5U or Bacillus subtilis R0179.

More preferably, a Pediococcus acidolactici strain is used, such as the Pediococcus acidolactici strain registered in the Collection Nationale de Cultures de Microorganismes (Microorganism Culture National Collection)(CNCM) in the Institut Pasteur de Paris (The Paris Pasteur Institute) under number MA 18 5 M.

Lactobacillus rhamnosus ATCC 53103, Weissella hellenica JCM10103, Bacillus cereus ATCC 14893, Bacillus cereus NCIB 40112 strains can also be used.

Preferably, the prophylactic composition used according to the invention comprises 10⁴ to 10⁷ colony forming units (cfu) of said bacterium of the Bacilli class per gram of the composition.

Preferably, the fish belonging to the salmonid class is selected amongst salmons, trout, char fish, grayling, and coregons.

According to a first preferred embodiment, the prophylactic composition used according to the invention comprises a food additive composition intended for fish belonging to the salmonid family.

Another object of the invention is to provide a method for preparing a food additive composition for salmonid fish characterized in that it comprises a step wherein cells are incorporated of a strain or several strains of bacteria of the Bacilli class into a carrier material physiologically compatible for food.

In this first embodiment, the food supplement or additive comprises cells of the strain(s) of bacteria belonging to the Bacilli class incorporated into a carrier, preferably, in the form of particles, more particularly, granules. The carrier wherein bacteria are incorporated can be of various natures. For example, said carrier can exclusively comprise or essentially consist in starch, including rice or corn starch, having been mixed with bacterial cells before being put in the form of particles, for example, granules.

The carrier wherein bacterial cells are incorporated can also exclusively comprise or essentially consist in β-cyclodextrin, the β-cyclodextrin being mixed with bacterial cells prior to a drying step, followed by a preparing step of particles, in particular granules. Preparing a food additive for fish comprising a β-cyclodextrin based carrier, as well as its manufacture in the form of granules, is disclosed, in particular, in U.S. Pat. No. 5,229,146.

For example, for preparing a food composition, also referred to as a food additive, the man skilled in the art can mix β-cyclodextrin with an appropriate amount of bacterial cells, so as to obtain a composition comprising 10³ to 10⁸ colony forming units of said bacterium or bacteria per gram of the final composition containing β-cyclodextrin, followed by submitting the thus obtained composition to a drying step, before producing particles, in particular granules, according to conventional methods.

Preferably, the carrier can be composed of calcium carbonate, eventually, combined with lactose.

The carrier used for preparing a prophylactic food additive composition according to the invention could also exclusively consist of or essentially consist of lactose, eventually in combination with sodium aluminosilicate, to said combination a culture medium could be added of the strain or a mixture of Lactobacillaceae strains, being previously submitted to a drying step.

In another aspect, for preparing a food prophylactic composition according to the invention, the man skilled in the art can use commercial carriers, such as the <<Ecoweaner®>>, <<Ecostart®>> carrier compositions marketed by the BIOMAR S.A company. (France), or also the <<Néostart® 7>>, <<Néoprima®>>, <<Néoextra®>>, <<Néoultra®>>, <<Néonova®>>, <<Néoastra>> or <<Néosupra®>> compositions, marketed by LE GOUESSANT company (France) or also the <<Nutra HP®>>, <<Classic®>>, <<Select®>> <<Royal Optima®>> carrier compositions marketed by TROUW company (France).

In this another aspect, the man skilled the art adds a given weight to one of said commercial compositions, preferably in the form of a culture medium for bacteria and containing said Bacilli cells to a known cfu value, in an amount selected so as to obtain a final mixture composition at a chosen concentration, of 10⁴ to 10⁷ colony forming units (cfu), per gram of the final mixture composition.

Using cells of at least one bacterium strain of the Bacilli class according to the invention is also characterized in that the composition being prepared consists in a whole food for fish belonging to the salmonid class.

According to still another aspect, the food additive prophylactic composition according to the invention can be added or mixed to a conventional food composition adapted to the various growth phases of the involved salmonid. For example, 10 parts in weight of the prophylactic composition according to the invention can be mixed to 300 parts in weight of a commercial food composition for fish of the salmonid family, so as to obtain a whole food for salmonids having a prophylactic effect against the vertebral compression syndrome.

Preferably, a food prophylactic composition according to the invention is distributed, in appropriate amounts depending on the number of fish present in the concerned fish farming pond, as soon as the larva's mouth opening or optionally, still as from the young fish stage (10 days at 10° C. after hatching).

According to the invention, a prophylactic treatment method is implemented for the vertebral compression syndrome in salmonids, characterized in that it comprises a step during which a food additive composition such as defined hereinabove is distributed, in appropriate daily amounts, in a fish farming pond where the salmonids are located.

Preferably, the period of time during which the food additive prophylactic composition is administered, or also the food composition containing said food additive prophylactic composition is at least three months, preferably, at least four months, and most preferably, at least five months.

According to a second embodiment of a prophylactic composition, the use according to the invention is characterized in that the composition being prepared consists in a pharmaceutical composition intended for fish belonging to the salmonid family.

For preparing such a pharmaceutical composition, the man skilled in the art can refer to the conventional techniques for preparing a pharmaceutical formulation mixing one or more excipients with the appropriate amount of strain bacterial cells or a mixture of bacterial strains of the Bacilli class.

For example, the excipient carrier can consist in lactose, to which the appropriate amount of bacterial cells is added.

The invention is further illustrated, without thereby being limited to, the following figures and examples.

### FIGURES

FIG. 1 shows X ray radiophotography of vertebrae of a spinal column of a rainbow trout not affected by the vertebral compression syndrome (FIG. 1A) and a X ray radiophotography of malformed and fused vertebrae of the spinal column of a rainbow trout suffering from the vertebral compression syndrome (FIG. 1B).

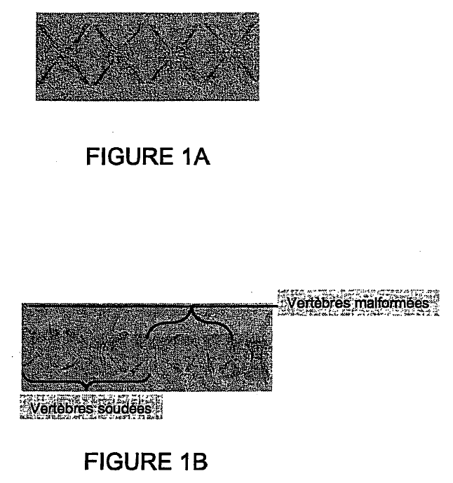

FIG. 2 shows the average death rate per week in a rainbow trout fish farm fed with (i) a standard food (solid diamond), (ii) a standard food and a florphenicol antibiotic supplement (grey shaded solid square), (iii) a standard food and a nutritional prophylactic composition according to the invention administered for a period of time of twenty days (star) or five months (solid circle).

On the ordinates, there is the number of deaths per week; on the abscissa, the farming period of time, expressed in number of weeks.

FIG. 3 illustrates the comparative results of the growth measurement of young fish controls and young fish having received a food additive composition according to the invention for 20 days (FIG. 3A) or for 5 months (FIG. 3B).

On the abscissa, there is the farming period of time (dates); on the ordinates, the young fish average weight, expressed in grams.

FIG. 4 illustrates the frequency of fish having vertebral compression, depending on their food diet. <<TEM>>=diet of fish with a standard food; <<NUF>>=diet of fish with a standard food added with the florphenicol antibiotic; <<B20>> and <<B05>>=fish fed with a standard food to which is added a prophylactic composition containing Lactobacillaceae of a Pediococcus acidilactici strain, respectively for a period of time of twenty days (B20) or five months (B05).

On the ordinates, there is the percentage of fish suffering from the vertebral compression syndrome.

### EXAMPLES

### Example 1

### Method for Preparing a Nutritional Prophylactic Composition According to the Invention

A. Materials and Methods

The method for preparing a food prophylactic composition can comprise adding cells of the strain(s) of the Bacilli class selected during preparing commercial food carriers.

After the cooking-extrusion phase, the still hot granulated base is coated through mixing with tepid fish oil (30° C.). Such oil can contain a food additive particle suspension, comprising the probiotic and the carrier thereof, in a concentration allowing to achieve the final seeding purpose.

B. Results of the Bacteriological Analysis

The food additive prophylactic composition prepared according to the hereinabove section <<Material and Methods>> was analyzed for its content in living Pediococcus acidilactici strain cells. The results are presented in the hereinbelow table I.

TABLE I
Checking of the food seeding

| Sample | Expected amount | | Measured amount | | Difference |
|---|---|---|---|---|---|
| Designation | cfu/g | log | cfu/g | log | log |
| Ecoweaner ® | 1.50 × 10⁶ | 6.18 | 2.50 × 10⁶ | 6.40 | +0.222 |
| n° 003 + | | | | | |
| Bactocell ® | | | | | |
| Ecostart ® | 1.50 × 10⁶ | 6.18 | 1.30 × 10⁶ | 6.11 | −0.062 |
| 15 n° 01 + | | | | | |
| Bactocell ® | | | | | |
| Ecostart ® | 1.50 × 10⁶ | 6.18 | 1.10 × 10⁶ | 6.04 | −0.135 |
| 15 n° 02 + | | | | | |
| Bactocell ® | | | | | |
| Ecostart ® | 1.00 × 10¹ | 1.00 | 1.00 × 10¹ | 1.00 | 0.000 |
| 15 n° 02 | | | | | |

The thus prepared prophylactic composition does contain the wanted P. acidolactici viable germs, showing the efficiency of the product.

### Example 2

### Comparative Study of Rainbow Trout Fish-Farming with a Food Prophylactic Composition According to the Invention and Other Food Compositions

A. Materials and Methods

A.1 Fish Farming Protocol of Rainbow Trout

Autumn strain rainbown trout (INRA), in the food starting phase, are distributed in six groups of three distinct batches, each batch comprising 500 10 day old young fish respectively:

(a) three control batches, exclusively fed with a standard food, the <Ecostart®>> food composition marketed by the BIOMAR SA company, the batch being designated as <<TEM>>;

(b) three batches fed with the Ecostart® standard foodstuff and treated using the Florphenicol antibiotic (Nuflor® veterinary pharmaceutical speciality) for 10 days as soon as the first feeding phase at a level of a Florphenicol daily amount of 18 mg per kg of body weight, such batches being designated as <<NUF>>;

(c) three batches fed with the standard food composition supplemented with a food prophylactic composition according to the invention, marketed under the Bactocell® trade mark by the Lallemand company, for twenty days as soon as the first feeding phase, such batches being designated <<B20>>; and

(d) three batches fed with the standard food composition are supplemented with the Bactocell® composition for five months as soon as the first feeding phase, such batches being designated <<B05>>.

A.2 Analyzed Parameters

On the above-mentioned various fish batches, the following parameters were respectively checked:

fish death rate;

the fish growth, through randomly taking 4 times at various moments 50 fish from each batch, and measurement of their total weight;

the digestive flora on all the (a) through (d) batches at a 20 day time and five months after the start of the trial. After 12 h of fasting three young fish were taken from each pond. After disinfection of the abdomen using alcohol at 70°, the distal part of the alimentary tract was separated through dissection in sterile conditions. After weighing the trial sample, the section of the alimentary tract was ground in a glass homogenizer containing 4.5 mL of Ringer solution. The ground material was diluted up to 1000 times through 1/10th successive dilutions. 1/10th and 1/1000th dilutions were applied to Petrifilm for counting the aerobic flora (7 day incubation at 11° C.);

the survival of Pediococcus acidilactici cells in the fish alimentary tract at day twenty, day thirty and after five months after the trial start. The ground material of the alimentary tract prepared according the herein described technique was also used for counting probiotics. Depending on treatments (a) through (d), appropriate dilutions were applied on MRS gelose for counting Pediococcus acidilactici (incubation for 48 hours at 45° C.), and on Sabouraud gelose supplemented with Chloramphenicol (50 mg L⁻¹), polymyxin B sulfate (1,6 mg L⁻) and amoxicillin (2.5 mg L⁻¹) for counting Saccharomyces cerevisiae (incubation for 48 hours at 30° C.);

the frequency and the severity of the vertebral compression syndrome on batches (a) through (d), through radiography on X rays on fifty fish of each of the batches.

B. Results

B.1. Death Rate

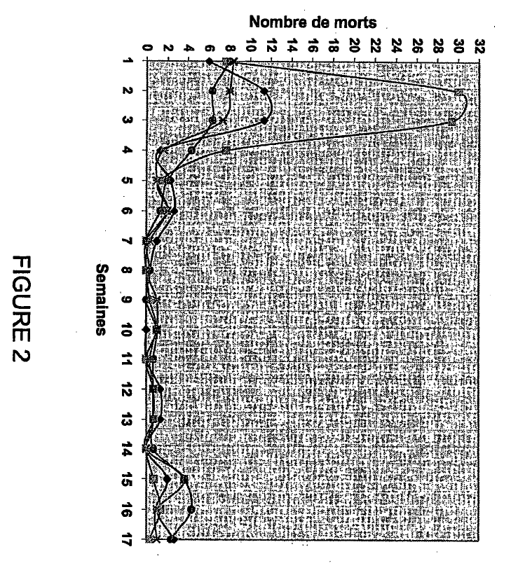

The analysis results of the weekly average death rate of fish belonging to batches (a) through (d) are represented on FIG. 2.

The results show that, during the first trial weeks, the death rate of the NUF batches (treatment with the florphenicol antiobitic) is particularly high, such an antibiotic most likely having toxic properties for the fish.

On the contrary, no significant difference was observed on the fish death rate respectively in the (a) control batch and in lot (d) the food of which was supplemented by a composition based on Pediococcus acidilactici (batches (c) and (d)).

A trend was however observed to a better survival of fish in batch (d) treated with a food supplement, in the absence of an antibiotic.

B.2. Analysis of the Young Fish Growth

The growth of all the batches of young trout (a) through (d) was normal. No statistically significant difference was observed regarding the growth of young fish on the various batches.

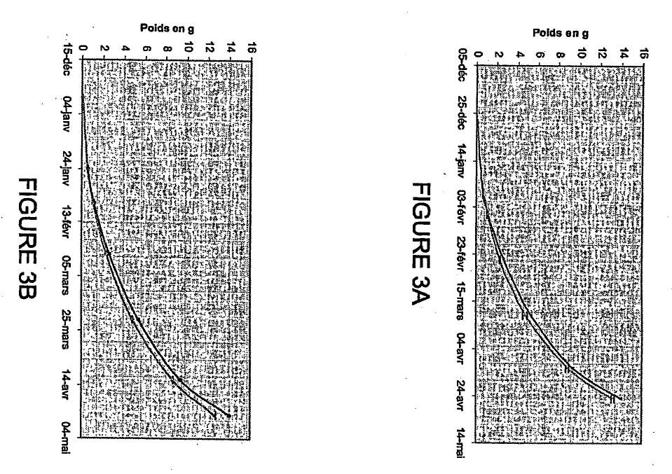

The results are illustrated on FIG. 3.

The results of FIG. 3 show that the young fish having received a food additive composition according to the invention have a growth identical to that of young fish in the control batch.

B.3 Analysis of the Digestive Flore

At day 20 after the trial start, the counts of the total cultivable germs are quite varied, and no statistically significant difference was observed depending on the batches.

The amounts of Pediococcus acidilactici cells, after twelve hours of fasting, are relatively low for fish from batch (c); they are equal to 2.1×10³±1.2×10³ cfu per gram. In batch (c), where the nutritional supplementation was stopped at day 20, no more Pediococcus acidilactici cell (batch (c)) was found. There is consequently no persistence of germs in the fish alimentary tract, after the treatment with the nutritional supplement was stopped.

At five months after the trial start, no significant difference was observed in the counts of the total cultivable germs, respectively:

control batch (a): 1.4×10⁷±0.8×10⁷ cfu per gram;

Pediococcus batch (d): 6.4×10⁴±2.4×10⁴ cfu per gram.

No Pediococcus acidilactici cell was found in the fish alimentary tract after twenty-four hours of fasting, whatever the batch considered. Such an observation confirms the non implantation of the probiotics in the alimentary tract.

These results also show that the prophylactic composition of nutritional supplement according to the invention exerts a regulating effect on the fish digestive flora.

B.4. Occurrence Frequency of the Vertebral Compression Syndrome

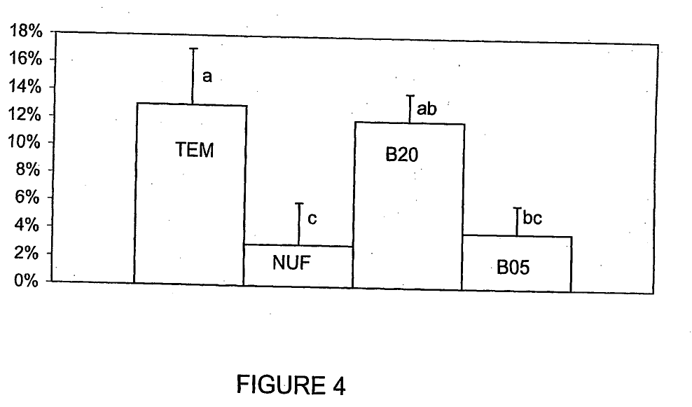

The occurrence frequency of the vertebral compression syndrome was analyzed in each of batches (a) through (d). The results are represented on FIG. 4.

As the results on FIG. 4 show, the treatment of the fish using the prophylactic composition of the nutritional supplement according to the invention, based on lactobacilles Pediococcus acidilactici cells and after a five month period treatment, considerably reduces the frequency of fish with vertebral compression syndrome, such a reduction being of the same order as that observed with the florphenicol antibiotic.

The results as set forth hereinabove enhance the interest of using cells of at least one bacterium strain of the Bacilli class for preparing a composition intended for preventing the vertebral compression syndrome in salmonids, more particularly in rainbow trout.

Thus, the use, as a food additive, of a prophylactic composition according to the invention seems well adapted to the drastic reduction of the occurrence of the vertebral compression syndrome in salmonids, without leading to numerous disadvantages linked to the use of an antibiotic compound. Such a composition used according to the invention is indeed well adapted to the present sanitary context in fish breeding farm and is much less risky for the fish population than using antibiotics on a preventive basis.

In addition, a prophylactic composition according to the invention is characterized by its implementation ease and its reduced cost. The profitability in salmonid fishing breedings in fish farms is higher.

1-9. (canceled)

10. A method for preventing vertebral compression syndrome in a fish belonging to the salmonid family, comprising administering to said fish at least one bacterium strain of the Bacilli class.

11. The method according to claim 10, characterized in that said composition comprises 10³ to 10⁸ colony forming units (cfu) of said bacterium of the Bacilli class per gram of the composition.

12. The method according to claim 11, characterized in that said composition comprises 10⁴ to 10⁷ colony forming units (cfu) of said bacterium of the Bacilli class per gram of the composition.

13. The method according to claim 10, characterized in that said bacterium strain(s) of the Bacilli class is (are) selected amongst bacteria of the Lactobacillaceae, Carnobacteriaceae, Enterococcaceae, Leuconostocaceae and Streptococcacea families.

14. The method according to claim 10, characterized in that the bacterium strain(s) of the Bacilli class is selected amongst the Bacillus subtilis, Bacillus cereus, Bacillus megaterium, Bacillus polymyxa, Bacillus licheniformis, Lactobacillus rhamnosus, Lactobacillus plantarum, Lactobacillus casei, Lactobacillus acidophilus, Lactobacillus johnsonii, Lactobacillus helveticus, Lactobacillus bulgaricus, Lactobacillus fructivorans, Lactobacillus brevis, Pediococcus acidilactici, Pediococcus pentosaceus, Carnobacterium divergens, Carnobacterium inhibens, Enterococcus faecium, Leuconostoc cremoris, Weissella hellenica, Streptococcus thermophilus, and Lactococcus lactis strains.

15. The method according to claim 10, characterized in that the fish belonging to the salmonid family is selected amongst salmons, trouts, char fishes, graylings and coregons.

16. The method according to claim 10, characterized in that said composition consists in a food additive composition for fish belonging to the salmonid family.

17. The method according to claim 16, characterized in that said composition consists in a whole foodstuff for fish belonging to the salmonid family.

18. The method according to claim 10, characterized in that said composition consists in a pharmaceutical composition for fish belonging to the salmonid family.

19. A method for inhibiting vertebral compression syndrome in a fish belonging to the salmonid family, comprising administering to said fish at least one bacterium strain of the Bacilli class.

20. The method according to claim 10, characterized in that said composition comprises 10³ to 10⁸ colony forming units (cfu) of said bacterium of the Bacilli class per gram of the composition.

21. The method according to claim 11, characterized in that said composition comprises 10⁴ to 10⁷ colony forming units (cfu) of said bacterium of the Bacilli class per gram of the composition.

22. The method according to claim 10, characterized in that said bacterium strain(s) of the Bacilli class is (are) selected amongst bacteria of the Lactobacillaceae, Carnobacteriaceae, Enterococcaceae, Leuconostocaceae and Streptococcacea families.

23. The method according to claim 10, characterized in that the bacterium strain(s) of the Bacilli class selected amongst the Bacillus subtilis, Bacillus cereus, Bacillus megaterium, Bacillus polymyxa, Bacillus licheniformis, Lactobacillus rhamnosus, Lactobacillus plantarum, Lactobacillus casei, Lactobacillus acidophilus, Lactobacillus johnsonii, Lactobacillus helveticus, Lactobacillus bulgaricus, Lactobacillus fructivorans, Lactobacillus brevis, Pediococcus acidilactici, Pediococcus pentosaceus, Carnobacterium divergens, Carnobacterium inhibens, Enterococcus faecium, Leuconostoc cremoris, Weissella hellenica, Streptococcus thermophilus, and Lactococcus lactis strains.

24. The method according to claim 10, characterized in that the fish belonging to the salmonid family is selected amongst salmons, trouts, char fishes, graylings and coregons.

25. The method according to claim 10, characterized in that said composition consists in a food additive composition for fish belonging to the salmonid family.

26. The method according to claim 16, characterized in that said composition consists in a whole foodstuff for fish belonging to the salmonid family.

27. The method according to claim 10, characterized in that said composition consists in a pharmaceutical composition for fish belonging to the salmonid family.
